(19) United States
(12) Patent Application Publication
(10) Pub. No.: US 2012/0266891 A1
(43) Pub. Date: Oct. 25, 2012

(54) SYSTEM FOR TRANSTRACHEAL ADMINISTRATION OF OXYGEN

(75) Inventors: Daniele Resca, San Felice Sul Panaro (IT); Giuseppe Zucchi, San Possidonio (IT); Alessandra Pedarzini, Finale Emilia (IT)

(73) Assignee: COVIDIEN AG, Neuhausen am Rheinfall (CH)

(21) Appl. No.: 13/496,726

(22) PCT Filed: Jul. 27, 2010
(86) PCT No.: PCT/EP2010/060866

§ 371 Date: May 25, 2012

(30) Foreign Application Priority Data
Oct. 1, 2009 (EP) 09171937.7

(51) Int. Cl. A61M 16/04

(52) U.S. Cl. 128/207.14

(57) ABSTRACT

A system for transtracheal administration of supplemental oxygen to a patient in need of continuous long tem oxygen therapy comprising a cannula (1), a transtracheal catheter (3) and an absorbing preformed disk (30), placed under a fixing flange (11) in order to absorb possible secretions.

### TECHNICAL FIELD

The invention concerns a system for transtracheal administration of supplemental oxygen.

In particular, the invention relates to a system for transtracheal administration of supplemental oxygen to a patient in need of continuous long term oxygen therapy (usually COPD—Chronic Obstructive Pulmonary Disease—Patients).

### BACKGROUND ART

In standard treatment for patients requiring supplemental oxygen it is still common to deliver oxygen from an oxygen source by means of devices such as nasal cannulas. Such treatment, however, requires a large amount of oxygen which is a waste and can cause irritation to the nose.

In order to overcome these drawbacks, it is known to supply supplemental oxygen to a patient through a transtracheal catheter instead of the cited commonly used nasal cannulas.

Various types of transtracheal catheter systems have been used in the medical field for many years to provide oxygen therapy to patients.

A first known catheter system consists of a catheter inserted in a flange used to secure the catheter to the patient's neck by means of a chain.

This and other known systems for transtracheal administration of supplemental oxygen have some drawbacks.

For example, some of these drawbacks are related to:

frequent dislodgements of the catheter,

risk of kinking of the catheter near the stoma,

bad sealing between the stoma and the catheter,

risk of injury of posterior tracheal wall during transtracheal catheter insertion.

### DISCLOSURE OF THE INVENTION

It is an object of the present invention to overcome the drawbacks above.

This object is achieved by a system for transtracheal administration of supplemental oxygen according to any of the claims below.

### BRIEF DESCRIPTION OF THE DRAWINGS

The system is shown in the following drawings, given purely by way of a non-limiting examples, in which:

FIGS. 1 to 16 show respectively schematic views of different elements of the system of the present invention;

FIG. 17 shows schematically, with some sectioned parts, the system of the present invention in use, applied in a human trachea, by way of examples.

### DETAILED DESCRIPTION OF THE PREFERRED EMBODIMENTS OF THE INVENTION

According to the drawings, the system of the present invention is composed by a plurality of elements/devices enabling administration of oxygen from a fixed or portable supply unit to the lungs of a patient through a stoma previously formed on the trachea by means of a surgical or percutaneous technique.

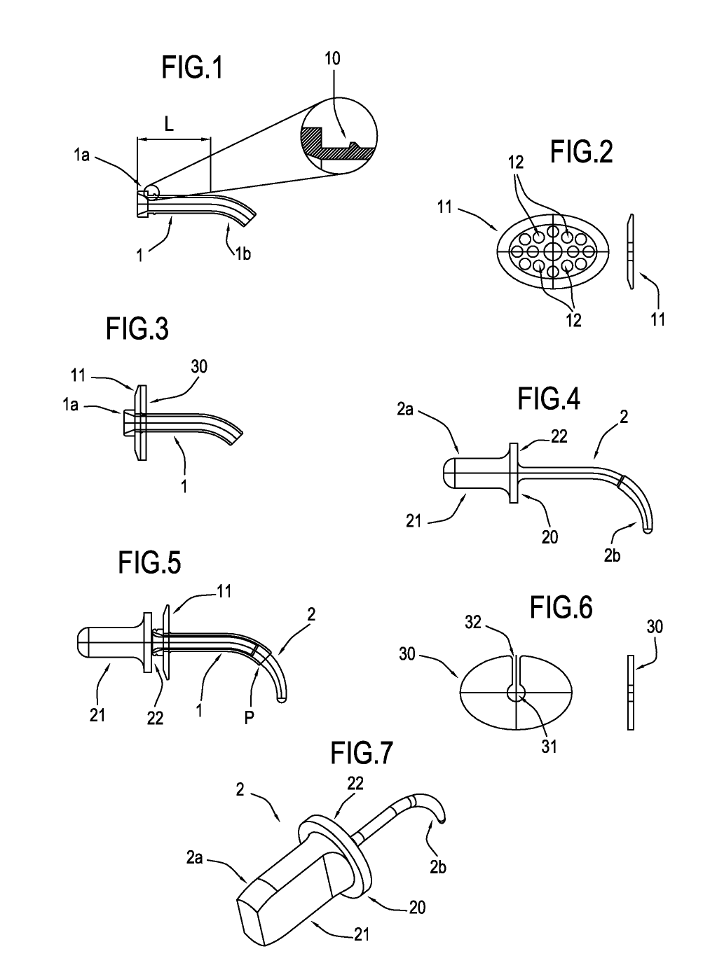

As shown in FIGS. 1-3, the system comprises a cannula 1, a fixing flange 11 and an absorbing disk 30.

The cannula 1 is essentially a conduit and presents a proximal end 1a and a distal end 1b.

Near its proximal end 1a, the cannula 1 comprises a shoulder 10 that defines blocking means to engage the fixing flange 11.

The fixing flange 11 supports thus the cannula 1 and presents a plurality of holes 12 provided to improve transpiration of the tissues that this part covers during use.

The absorbing preformed disk 30 is placed under the fixing flange 11, in order to increase transpiration and absorb possible secretions leaching from the interspace between the stoma and the cannula 1.

As shown in FIG. 6, the absorbing preformed disk 30 presents an internal hole 31 which size is similar to the external diameter of the cannula 1. The disk 30 also presents a radial cut 32 to allow its easy insertion on the cannula 1.

The system further comprises an insertion guide 2 for the cannula 1 and a transtracheal catheter 3.

Advantageously the guide 2 is made of a flexible soft material.

The guide 2 presents a proximal end 2a and an opposite distal end 2b.

The proximal end 2a defines handle and identification means 21 for the positioning of the thumb and the forefinger.

The guide proximal end 2a furthermore provides a stop surface 22 for the cannula 1.

The transtracheal catheter 3 to be used is better described in a different patent application filed in the name of the same Applicant.

Advantageously the transtracheal catheter 3 is pre-shaped, according to a reversed U shape, for matching with the cannula 1.

The system further comprises an adhesive patch 40 designed to fix the cannula 1 to the patient skin.

The devices or kits forming the system are better described below following the procedure sequence.

Once the stoma has been created, usually in the interspace below the cricoid and before the first tracheal ring, a cannula 1 is positioned inside the stoma through the aid of a guide 2.

The cannula 1 is preferably formed by plastic material whose hardness is sufficient to keep constant its internal diameter in any positioning of the neck (bending or twisting movements).

The cannula 1 serves as passage conduit and housing device for a transtracheal catheter 3 and for the formation of a mature stoma having a controlled size.

To satisfy all the different requirements in terms of tissues thickness range, from the epidermis to the internal front wall of the trachea, a plurality of cannulas 1, each one having a different length will be provided, each one marked with the relevant length size code.

Advantageously this variable length is marked with the letter L on the cannula 1.

The distal end 1b of the cannula 1 has a curved shape suitable for guiding the proper advancement of a transtracheal catheter 3 in the central part of the trachea, preventing damage to the trachea backwall.

As already explained above, the fixing flange 11 presents a plurality of holes 12 provided to improve transpiration of the tissues that this part covers during use.

FIG. 3 shows the cannula 1 assembled with a fixing flange 11 and an absorbing disk 30 described in detail below.

In use, the guide 2 is then inserted in the cannula 1 suitable for the patient undergoing the procedure (the physician usually get this information observing how deep the graduated needle has been inserted to puncture the front wall of the trachea in percutaneous technique).

The handle and identification means 21 allows easy handling and easy identification of the guide 2 and the cannula 1 orientation during insertion manoeuvre, helping to keep a good control of the cannula 1 positioning (caudal positioning).

The cannula 1 is then pushed towards the stop surface 22 located at proximal end 2a of the guide 2, allowing a total control of the cannula 1 positioning during the insertion.

The distal end 2b of the guide 2 is tapered and curved with an atraumatic spheric/ball tip. The external diameter of the guide 2, covering the length from the surface 22 to a point P (shown in FIG. 5) in the curved part, combines with the internal diameter of the cannula 1 with a narrow backlash.

The guide 2 carrying the cannula 1 is then inserted in the stoma.

The insertion shall be made with the guide 2 oriented vertically, with the curved part in the caudal position.

Once placed the cannula 1 in the correct position, removal of the guide 2 is performed while keeping fixed the flange 11 with two fingers of one hand, preferably forefinger and middle finger, while with the other hand the guide 2 is extracted from the stoma.

The absorbing preformed disk 30 is placed under the fixing flange 11.

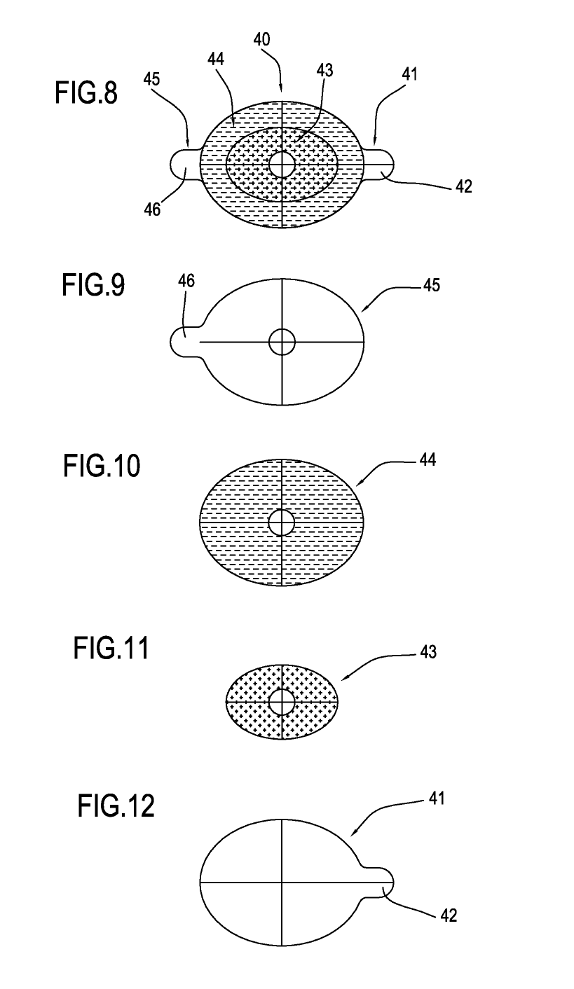

The following steps are used in fixing the cannula 1 by means of the adhesive patch 40, shown in FIG. 8, properly designed for this purpose.

The adhesive patch 40 comprises a plurality of assembled preformed layers.

FIGS. 8 to 12 show a preferred not limiting embodiment of the patch 40 and its preformed layers.

In detail, the adhesive patch 40 comprises:

an antiadhering paper support 41 having a tab 42 for easy removal of the same before the patch positioning (FIG. 12);

a film 43 with a central hole and a plurality of distributed microholes having the purpose of avoiding adhesion with the fixing flange 11 (FIG. 11);

a transparent adhesive transpirant film 44, allowing adhesion to the patient skin in an annular area around the fixing flange 11 (FIG. 10), the film 44 having a central hole;

a support film 45 for supporting the transparent adhesive film 44 and able to give due consistency to the patch 40 during application, the layers 43 and 44 being not selfstanding (FIG. 9); the support film 45 has a tab 46 for its easy removal.

The respective central hole of any of said layers 43, 44 and 45 has a diameter size slightly increased with respect to the external diameter of the proximal end 1a of the cannula 1.

The film 43 has the same shape as the fixing flange 11, in order to avoid the adhesive film 44 to adhere on the flange 11 and to allow the adhesive film 44 to adhere only in a peripherical annular area. This fact makes the procedure of replacing the patch easy, without risk of withdrawing the cannula 1 with consequent dislodgements and related inconveniences.

Once the cannula 1 is fixed to the skin of a patient by the adhesive patch 40, the transtracheal catheter 3 can be inserted inside the cannula 1.

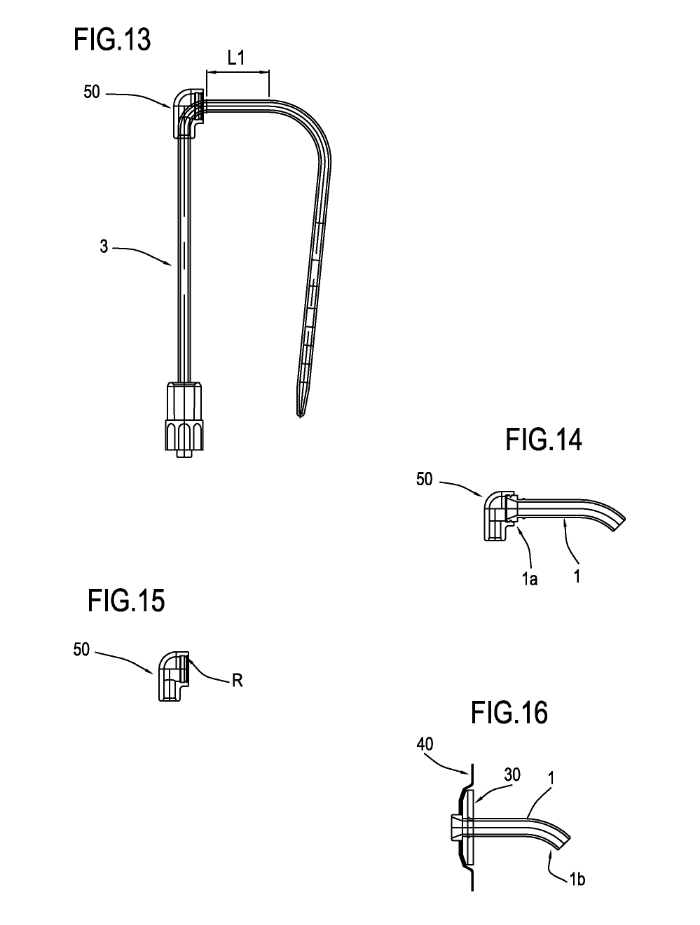

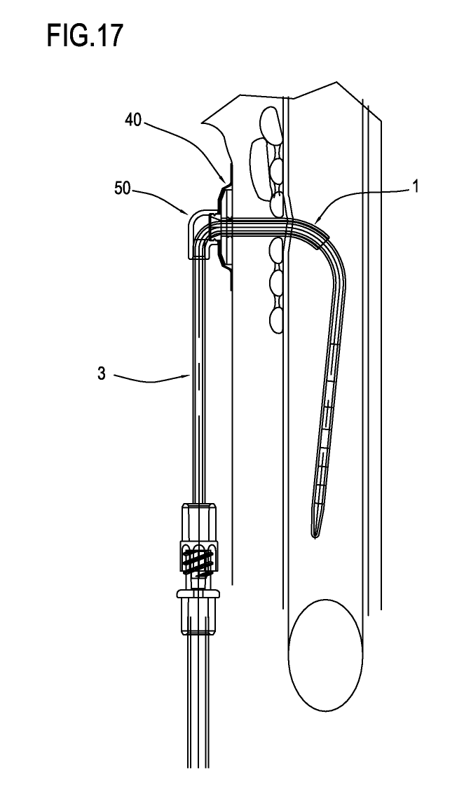

In detail, the variable length portion L of the cannula 1 (see FIG. 1) is supposed to match with the variable length portion L1 of the transtracheal catheter 3 (see FIG. 13).

Advantageously, thanks to the pre-shaping of the transtracheal catheter 3, the patient and/or his attendant are not entrusted with its positioning, which is otherwise bound by the mutual shape equivalence in the contact areas with the cannula 1.

Optimal fixing of the transtracheal catheter 3 is further ensured by an elbow connector 50, preferably made of soft rubber, sliding on the external surface of the transtracheal catheter 3 which is to be secured on the proximal end 1a of the cannula 1 by means of an inner gripping ring R (see FIGS. 14 and 15).

The elbow connector 50 ensures at the same time a sealing connection between the transtracheal catheter 3 and the cannula 1 itself preventing any fluid leakage from the trachea to the outside.

The system according to the present invention provides a plurality of advantages with respect to the prior art.

Some of the advantages that the system is able to contribute are described below.

In particular, the introduction of the cannula 1 as housing for the transtracheal catheter 3 with a specific pre-shaping and relevant transparent adhesive patch 40 allows:

a bound and univocal correct positioning of pre-shaped transtracheal catheter, allowing only very minor movements to the catheter itself—less catheter dislodgement—while in the traditional system the catheter is kept in place by a flange connected with a necklace;

improved fixing between the cannula and the patient skin through the use of the transparent adhesive patch in contrast to loosen necklace, increasing the comfort of the patient;

fixing means less visible than the necklace solution, increasing psychological acceptance by the patient;

to minimize the dragging force exerted on the stoma by the traditional transtracheal catheter, increasing the comfort of the patient;

to keep unchanged the stoma lumen (cannula rigid enough) thus avoiding any kinking of the transtracheal catheter in this specific area, increasing the safety of the patient;

better sealing between the stoma and the transtracheal catheter through the interposition of the cannula, thus achieving no leakage of oxygen from the stoma to the environment and less contact between the trachea and the external environment, and in this way increasing the safety of the patient with a reduced risk of cross contamination;

better management of the transtracheal catheter replacement, increasing usability and the comfort of the patient;

reduced risk of injury of posterior tracheal wall during transtracheal catheter insertion due to cannula shape guiding the insertion of the catheter to the correct position;

as a consequence of an easier transtracheal catheter replacement, the use of disposable transtracheal catheter to be changed on a daily basis; such a use is to be preferred in place of current state of the art reusable catheter with the burden of the cleaning procedure;

the further advantage of using the same cannula during the stoma creation up to when the stoma is completely healed, obtaining a mature stoma with a minimum diameter necessary to permit the therapy through transtracheal catheter.

In fact, it is known that most of the complications associated with the use of the traditional transtracheal system occurs during the latter phase once the stent is exchanged for the functioning catheter (SCOOP Catheter). Since the insertion tract is not yet mature the catheter may become dislodged easily and accidental displacement of the catheter usually occurs at night with severe consequences such as respiratory distress, hypoxiemia and subcutaneous emphysema.

The cannula 1 according to the system of the present invention is foreseen to be kept in situ ranging from one week up to several weeks in relation to patients needs, i.e. during healing of the stoma it may be necessary to clean and treat the affected part leading to a more frequent change need of the cannula.

The cannula 1 is advantageously foreseen disposable to avoid the patient the burden of cleaning it.

1. A system for transtracheal administration of supplemental oxygen comprising a transtracheal catheter; a cannula for guiding the catheter in the trachea; a fixing flange for supporting said cannula.

2. A system as claimed in claim 1, further comprising an absorbing preformed disk, placed under the fixing flange in order to absorb possible secretions leaching from an interspace between the stoma and the cannula.

3. A system as claimed in claim 1, further comprising an insertion guide for inserting and positioning the cannula.

4. A system as claimed in claim 3, wherein the insertion guide includes a distal end which is tapered and curved.

5. A system as claimed in claim 1, further comprising an adhesive patch designed to fix the cannula to patient skin.

6. A system as claimed in claim 5, wherein the adhesive patch comprises an antiadhering paper support having a tab for easy removal of the same before positioning of the adhesive patch.

7. A system as claimed in claim 5, wherein the adhesive patch comprises a film with a central hole and a plurality of distributed microholes having the purpose of avoiding adhesion with the fixing flange.

8. A system as claimed in claim 5, wherein the adhesive patch comprises a transparent adhesive film, allowing adhesion to the patient skin in an annular area around the fixing flange, said film having a central hole.

9. A system as claimed in claim 8, wherein the adhesive patch comprises a support film for supporting the transparent adhesive film and able to give due consistency to the adhesive patch during application, the support film having a tab for its easy removal.

10. A system as claimed in claim 1 wherein the fixing flange includes a plurality of holes provided to improve transpiration of tissues that this part covers during use.
